(19) United States
(12) Patent Application Publication
(10) Pub. No.: US 2022/0039717 A1
(43) Pub. Date: Feb. 10, 2022

(54) METHOD, SYSTEM AND APPARATUS FOR EVALUATING SENSORY ASSESSORS' CONCENTRATION ABILITY

(71) Applicant: CHINA NATIONAL INSTITUTE OF STANDARDIZATION, Beijing (CN)

(72) Inventors: Bolin SHI, Beijing (CN); Kui ZHONG, Beijing (CN); Houyin WANG, Beijing (CN); Lei ZHAO, Beijing (CN); Guilong CHEN, Beijing (CN); Lulu ZHANG, Beijing (CN); Ran XIE, Beijing (CN); Longyun LIU, Beijing (CN); Tianpeng HE, Beijing (CN)

(73) Assignee: CHINA NATIONAL INSTITUTE OF STANDARDIZATION, Beijing (CN)

(21) Appl. No.: 17/429,641

(22) PCT Filed: Oct. 10, 2019
(86) PCT No.: PCT/CN2019/110235

§ 371 Date: Aug. 10, 2021

(30) Foreign Application Priority Data
Aug. 23, 2019 (CN) 201910787410.8

(51) Int. Cl. A61B 5/16; G06K 9/62

(52) U.S. Cl. CPC A61B 5/168; G06K 9/623

(57) ABSTRACT

The attention recognition embodied by the method for evaluating the concentration ability of a sensory assessor is organically combined with evaluations for three categories of ranking capability, namely, excellent, good, and poor. Therefore, sensory assessors displaying high sensibility and poor attention form part of the group possessing excellent ranking capability, while sensory assessors exhibiting moderate sensibility and high attention can be found in the group possessing good ranking capability. Furthermore, sensory assessors displaying fair sensibility and high attention can be found in the group with poor ranking capability. This system can identify the concentration ability of assessors, therefore, providing support for the reliability of ranking results.

### CROSS REFERENCE TO THE RELATED APPLICATIONS

This application is the national phase entry of International Application No. PCT/CN2019/110235, filed on Oct. 10, 2019, which is based upon and claims priority to Chinese Patent Application No. 201910787410.8, filed on Aug. 23, 2019, the entire contents of which are incorporated herein by reference.

### TECHNICAL FIELD

The invention relates to the technical field of sensory analysis, and in particular, to a method, system, and apparatus for evaluating the concentration ability of sensory assessors.

### BACKGROUND

Sensory evaluation is a measuring technique for assessing the sensory characteristics of a product, such as its appearance, taste, smell, and texture using the sensory organs. Consequently, to guarantee the reliability, objectivity, and accuracy of a sensory evaluation result, it is necessary to scientifically present a reasonably prepared sample to a panel (machine) that passed screening, training, and examination for evaluation. The sensory evaluation is conducted to the test samples to obtain original evaluation data from each assessor using a scientific sensory analysis method (method), which is selected by an experienced sensory analyst (person). Then, the analyst subjects the data to statistical analysis to obtain the sensory quality of the product.

The sensory evaluation ranking method is used during the scaling and classification process is a rating technique requiring sensory assessors to rank a series of samples according to the strength of specific sensory characteristics. This method can be used to determine the influence of different materials, processing, treatments, packaging, storage, and various other conditions on the intensity of one or more sensory characteristics in a product. This technique can also be employed to perform prescreening before the intensive sensory evaluation (e.g., descriptive analysis) starts, as well as to screen and train sensory assessors.

The ranking method links the difference test with the descriptive analysis, meaning that assessors are only suitable for the difference test if they are unable to recognize the strength order of differences between products. Furthermore, assessors with satisfactory ranking capability may become descriptive analysts via further training.

Any measurement should be completed by a corresponding detection instrument. Additionally, the performance of the instrument directly determines the reliability, objectivity and accuracy of the result. The sensory evaluation instrument is represented by an evaluation panel composed of several assessors, while the original sensory ranking data is sourced directly from the evaluation results by sensory assessors. ideally, the expectation is that every assessor can provide a response that fully corresponds with either the actual quality order or the theoretical optimal order in each case. An answer that fails to reflect the real difference order of the sensory quality between various samples according to the corresponding experimental data of the sensory evaluation denotes the poor or unstable ranking capability of the sensory assessor, resulting in an unreliable experimental result and conclusion. in addition, the application and guidance of the conclusion in new product development, product improvement, raw material replacement, quality control, market forecasting, and a variety of other aspects will be affected accordingly. Therefore, the ranking capability of a sensory assessor is essential in obtaining a reliable and stable sensory ranking.

The evaluation technique for the ranking capability performance of a sensory assessor lies in the technical support to reflect the availability of “ranking instrument”. Therefore, this technique not only guides assessors in correcting and achieving the requisite accuracy before being employed but also assists them in performing periodic verification after a certain period, consequently, conforming to the norms and requirements of detecting and guaranteeing the validity or accuracy of the ranking result. This method presents the fundamental guarantee in achieving the value of the sensory ranking data, therefore, being a crucial means for reflecting the ranking test level of a sensory analysis laboratory and forming a significant part in the establishment and recognition of its ranking capability.

Therefore, the evaluation technique for the ranking capability performance of a sensory assessor in the sensory analysis laboratory can effectively control the “ranking instrument” to keep in good condition, achieving the reliability of the ranking data detected by the instrument. This process ensures that the requirements for sensor' analysis during scientific research, experimental execution, and production are met, while significantly promoting the wide application of the sensory ranking method, rendering the sensory analysis laboratory exceedingly significant.

Theoretically, there is a substantial correlation between the true ranking and repeated. ranking performance of a sensory assessor. Therefore, a highly trueness sensory assessor will also exhibit strong repeatability, while that of a sensory assessor with low trueness will be poor. However, during the actual evaluation process, some situations contradict this assertion. Research shows that the reason for high trueness but poor repeatability lies in an attitude problem, namely, a lack of attention and seriousness in ranking the experimental samples rather than being an issue of capability, an error in the preparation and presentation of experimental samples, or an incorrect ranking evaluation method. Therefore, the result fails to reflect the normal level (highly trueness and strong repeatability) of these sensory assessors. Care should be taken when employing these types of sensory assessors, since their reliability regarding maintaining a serious and professional attitude during experiments is uncertain, potentially causing an ambiguous situation not conducive to obtaining a reliable experimental result. in the case of sensory assessors with a moderate trueness ranking capability and excellent repeatability, it fully reflects their serious attitude and stability while ranking experimental samples. Therefore, although these sensory assessors are usually reliable and practical and are frequently employed during sensory evaluation experiments, some of them have potential morn for improvement. Consequently, it is essential to evaluate the attention and concentration ability of a sensory assessor, but no system exists in the prior art for the rapid analysis of these attributes by means of computer software.

### SUMMARY

The invention aims to provide a method for evaluating the concentration ability of a sensory assessor, which can solve the lack of guidance in the prior art.

These objectives are achieved via the following technical scheme of the invention:

A method for evaluating the concentration ability of a sensory assessor is composed of the following steps:

S1, entering the first kind of data into a data input unit and saving it to a storage unit;

S2, processing the first kind of data with a data processing unit to obtain the second kind of data, the third kind of data, the fourth kind of data, and the fifth kind of data;

S3, analyzing the second kind of data, the third kind of data information, the fourth kind of data and the fifth kind of data information with a data analysis unit, therefore, recognizing the concentration ability of a sensory assessor; and

S4, displaying serial numbers denoting the sensory assessors with adequate concentration ability in a result display unit.

The first kind of data refers to the ranking information obtained by an assessor by repeatedly ranking the sensory quality of n samples at different concentrations for the m rounds where n=6 and m=12.

The data processing unit includes a ranking capability classification module, a true ranking capability module, a repeated ranking capability module, and a ranking focusing capability module. Specifically, the processing steps of the data processing unit are as follows:

Firstly, the value of a Spearman rank correlation coefficient rₛ for each round of ranking by each sensory assessor is calculated by the ranking capability classification module according to the ranking information. Then a median and a mode of the values of the Spearman rank correlation coefficients rₛ are obtained after m rounds of ranking experiments by each sensory assessor.

The Spearman rank correlation coefficient rₛ is calculated according to the following formula:

$${r}_{s} = 1 - \frac{6 {\sum }_{i = 1}^{n} {d}_{i}^{2}}{n ( {n}^{2} - 1 )} \qquad ( 1 )$$

where rₛ is the rank correlation coefficient; n is the number of ranking experiment samples; dᵢ is the difference between the real rank and the rank of the i^{th} sample determined by the sensory assessor during the ranking experiment.

For example, it is preferable that when n=6, the sensory assessors with a mode=1.00 belong to the first kind of sensory assessor group that possesses excellent ranking capability; the sensory assessors with a median=0.943 belong to the second kind of group possessing good ranking capability, and the remaining sensory assessors belong to the third kind of group possessing poor ranking capability.

Then, the true ranking capability of a sensory assessor is evaluated using the true ranking capability module, eliminating a result for a round with an rₛ value of less than 0.60 among the m rounds of ranking by each sensory assessor. Then the rank data rₛ value for each remaining round of ranking is converted into a corresponding equidistant data value via the Fisher conversion. Then, arithmetic mean value Z̅ᵣ of the Zᵣ values is obtained for the remaining rounds after eliminating abnormal experiments for each sensory assessor. Therefore, the higher the Zᵣ value, the higher the correct ranking capability, while the Zᵣ Fisher conversion is used to convert the rₛ value for each ranking experiment of each sensory assessor into a Zᵣ value according to the following calculation formula:

$${Z}_{r} = {tanh}^{- 1} ( {r}_{s} ) = {\sum }_{N = 0}^{∞} \frac{{r}_{s}^{2 N + 1}}{2 N + 1} \qquad ( 2 )$$

where rₛ is a rank correlation coefficient, and N is the number of terms in the inverse hyperbolic tangent expansion.

The value of Z̅ᵣ is calculated according to the following formula:

$$\overline{{Z}_{r}} = \frac{{\sum }_{j = 1}^{m} ( {n}_{j} - 3 ) {Z}_{rj}}{{\sum }_{j = 1}^{m} ( {n}_{j} - 3 )} \qquad ( 3 )$$

where m is the number of evaluation repeats after eliminating abnormal experiments; nⱼ is the number of samples in the j^{th} repeated evaluation, and nⱼ is 6; the Zᵣⱼ value is the Fisher conversion Zᵣ value of the correlation coefficient rₛ value for the j^{th} repeated evaluation.

Then, the repeated ranking capability of a sensory assessor is evaluated by the repeated ranking capability module: calculating a standard deviation (S_{Zr}) of the Zᵣ values for the remaining rounds obtained after eliminating abnormal experiments for each sensory assessor; the repeated ranking capability of each sensory assessor is reflected according to the S_{Zr}; while the smaller the S_{Zr}, the higher the repeated ranking capability.

S_{Zr} is calculated according to the following formula:

$${S}_{{Z}_{r}} = \sqrt{\frac{{\sum }_{j = 1}^{m} {( {Z}_{{r}_{j}} - \overline{{Z}_{r}} )}^{2}}{m}} \qquad ( 4 )$$

where m is the number of evaluation repeats after eliminating abnormal experiments here; the Zrⱼ is the Fisher conversion Zᵣ of the correlation coefficient rₛ in the j^{th} repeated. evaluation; Z̅ᵣ is a mean value of the Zᵣ values obtained by applying the Fisher conversion to the rₛ values of the remaining rounds after eliminating abnormal experiments for a certain assessor.

This process is followed by calculating a ratio (CV value) of the S_{Zr} of Zᵣ values after multiple rounds of ranking to obtain the Z̅ᵣ value for each sensory assessor by the ranking focusing capability module. The CV value is calculated according to the following formula:

$$CV = \frac{{S}_{{Z}_{r}}}{\overline{{Z}_{r}}} \qquad ( 5 )$$

The second kind of data denotes the median and mode of an rₛ value; the third kind of data is the Z̅ᵣ value; the fourth kind of data is the S_{Zr} value, and the fifth kind of data is the CV value.

The data analysis unit is configured to analyze the CV value of each kind of sensory assessor group; when the CV value >20%, the first kind of sensory assessor group (with excellent ranking capability) is recognized as sensory assessors with high sensibility and poor attention; when the CV value ≤17%, the second kind of sensory assessor group (with good ranking capability) is recognized as sensory assessors with moderate sensibility and high attention; and when the CV value ≤21%, the third kind of sensory assessor group (with poor ranking capability) is recognized as sensory assessors with fair sensibility and high attention.

The serial numbers for a sensory assessor turn red in the result display module when they exhibit high sensibility and poor attention, yellow in the case of moderate sensibility and high attention, and green in the case of fair sensibility and high attention.

The invention further discloses a system for recognizing the concentration ability of a sensory assessor to be used in the evaluation method mentioned above. This system comprises a data input unit for entering the first kind of data; a storage unit for saving the first kind of data; a data processing unit for processing the first kind of data to obtain the second kind of data, the third kind of data, the fourth kind of data and the fifth kind of data; a data analysis unit for analyzing the second kind of data, the third kind of data, the fourth kind of data and the fifth kind of data , therefore, determining the concentration ability of a sensory assessor; and a result display unit for displaying the serial numbers denoting the concentration ability of sensory assessors.

A device comprising the above system for recognizing the concentration ability of a sensory assessor also falls within the protection scope of the present invention.

The invention presents the following advantages:

(1) The system can input and store the ranking result of each assessor at any time, and can, therefore, he retrieved and examined when necessary.

(2) The method can be used to analyze the ranking capability of the sensory assessor disobeying the rule of relevance between true ranking capability and repeated ranking capability, namely, attention recognition. The attention recognition embodied by the system of the present invention is organically combined with the evaluations of three categories of ranking capabilities, namely, excellent, good, and poor. Therefore, sensory assessors with high sensibility and poor attention are found in the group displaying excellent ranking capability, sensory assessors with moderate sensibility and high attention are found in the group displaying good ranking capability, and sensory assessors with fair sensibility and high attention are found in the group displaying poor ranking capability.

(3) An idea of standard deviation/mean ratio under multi-repetition, namely, an idea of variable coefficient, is introduced in the concentration processing in present invention. This system in particular can realize the conversion of the statistical data r value, which displays sequential characteristics and reflects a ranking result of each round, into a Zᵣ value with equidistant characteristic data using the Zᵣ Fisher conversion. Therefore, this system guarantees the implementation and application of the variable coefficient thought and assists in the scientific analysis of concentration,

Due to the advantages mentioned above, assessors with a high inherent ranking level but poor attitude can be accurately recognized, avoiding the potential risk associated with these assessors and unclear evaluation results. In contrast, assessors with an acceptable ranking level, a serious attitude and high stability can also be identified via the advantages mentioned above. These assessors are reliable, practical. Therefore, the experiment manager should pay more attention to them. Some of these assessors may have potential for improvement in their ranking ability. Consequently, these advantages provide support in obtaining reliable ranking results.

### BRIEF DESCRIPTION OF THE DRAWINGS

FIG. 1 is a structural diagram of a system for evaluating the concentration ability of sensory assessors.

FIG. 2 is a structural diagram of a data processing unit in one embodiment.

### DETAILED DESCRIPTION OF THE EMBODIMENTS

The present invention will be further specified by the detailed embodiments below However, it should be noted that the present invention may be implemented in various ways and should not be limited by the embodiments illustrated here. On the contrary, these embodiments are provided to render the present invention more apparent and complete, While fully conveying the invention scope to those skilled in the art.

The terms “comprise” or “include” mentioned throughout the description and claims are inclusive wording and should, therefore, be interpreted as “include but not limited to.” What is subsequently outlined in the description are preferred embodiments of the present invention, Which are aimed at the general principle of the description, but are not intended to define the present invention scope. The protection scope of the present invention shall be subject to the protection scope defined by the claims.

Unless expressly specified otherwise, the various methods employed in the present invention are conventional, while the different materials and reagents are commercially available.

### Embodiment 1

The method for evaluating the concentration ability of sensory assessors comprises the following steps:

S1, entering first kind of data to a data input unit and storing it in a storage unit;

S2, processing the first kind of data using a data processing unit to obtain the second kind of data, the third kind of data, the fourth kind of data, and the fifth kind of data;

S3, analyzing the second kind of data, the third kind of data, the fourth kind of data and the fifth kind of data using a data analysis unit, thereby determining the concentration ability of a sensory assessor;

S4, displaying serial numbers for the sensory assessors with specific attention characteristics in a result display unit.

The first kind of data refers to the ranking information obtained by an assessor by repeatedly ranking the sensory quality of n samples at different concentrations for the in rounds where n=6 and m=12.

The acquisition method of first kind of data is as follows:

1. The Screening of Assessors

For this process, 33 sensory assessors with a normal and relatively sensitive basic sense of taste (sour, sweet, hitter, and salty) were screened in accordance with the GB/T 12312-2012 Sensory Analysis Method of investigating Sensibility of Taste. Then, the screening continued by training the assessors based on the evaluation method and technical points of the skilled taste ranking experiment in accordance with the experimental requirements of the GB/T 12315-2008 Sensory Analysis Methodology: Ranking.

2. The Preparation of the Ranking Samples

Sucrose solution was selected as the ranking object of sweetness samples to evaluate the performance of ranking capability. Considering the negative emotion caused by sensory fatigue and multiple ranking repetitions, the overall concentration of the sweetness samples should be moderate (not too sweet, but sweet enough), The concentration difference among the samples of the ranking experiment series were set by referring to a threshold of the average sweetness difference in the panel of 33 sensory assessors. The extremely low concentration difference makes it challenging for assessors to distinguish the strength order of the sweetness, resulting, in disordered and incorrect ranking results from the majority of the sensory assessors, and losing the evaluation significance of ranking capability performance and, therefore, concentration differences that are too low should be avoided. On the contrary, the concentration difference should not be too high either, since this will allow the sensory assessors to correctly rank the strength order of sweetness too quickly, which also fails to be of any significant value during the ranking capability evaluation. The following basic principles are used for the preparation of the series of sample concentrations: ensure that a ¼ of the sensory assessors achieve an accurate ranking, while a ¼of the sensory assessors find it challenging, and the remaining ½ of the sensory assessors fail to obtain the correct order of an individual sample. Additionally, considering the dual factors of index increase in ranking difficulty caused by the increase of samples and the shortage of statistical significance caused by insufficient number of samples, the sweet solution at 6 concentrations were selected deliberately Specific concentrations are shown in Table 1.

TABLE 1
Sample rank and the corresponding concentrations

| Sensory | Correct rank and corresponding concentration (g · L⁻¹) | | | | | |
|---|---|---|---|---|---|---|
| characteristics | 1 | 2 | 3 | 4 | 5 | 6 |
| Sweetness | 15.2 | 18.0 | 21.3 | 25.1 | 29.6 | 34.9 |

3. Sensory Ranking Experiment

Sensory assessors were given a sweet solution at six different concentrations during each round of the experiment and requested to rank the sweetness strength of the solution from the weakest to the strongest based on sensory evaluation, with the weakest denoted by ranking No. 1 (rank), and the strongest signified by ranking No. 6, The samples where the strength was challenging to be distinguished, required different rank, avoiding allocation of the same rank to more than one sample, namely, a mode of forced-choice operation. Each sensory assessor requires 12 rounds of repeated ranking experiments in total, and all experimental samples are coded with three different random figures, while a randomized complete block design facilitates the providing order of the samples in each experiment.

Therefore, n=6 and m=12 generally denote the actual operational process, and the data has practical guidance significance.

The data processing unit includes a ranking capability classification module, a true ranking capability module, a repeated ranking capability module, and a ranking focusing capability module. Specifically, processing steps of the data processing unit are as follows:

First, a Spearman rank correlation coefficient r value is calculated for each round of ranking by each sensory assessor using the ranking capability classification module according to the ranking information. Then, a median and a mode for the values of the Spearman rank correlation coefficients r are obtained after the in rounds of the ranking experiments by each sensory assessor are calculated.

The Spearman rank correlation coefficient is calculated according to the following formula:

$${r}_{s} = 1 - \frac{6 {\sum }_{i = 1}^{n} {d}_{l}^{2}}{n ( {n}^{2} - 1 )} \qquad ( 1 )$$

where rₛ is a rank correlation coefficient n is the number of ranking experiment samples, and dᵢ is the difference between the real rank and the rank of the sensory assessor of the i^{th} sample during the ranking experiment.

For example, it is preferable that when n=6, the sensory assessors with a mode=1.00 belong to the first kind of sensory assessor group possessing excellent ranking capability; the sensory assessors with a median=0.943 belong to the second kind of group possessing good ranking capability, and the remaining sensory assessors belong to the third kind of group possessing poor ranking capability.

Then, the correct ranking capability of a sensory assessor is assessed using the true ranking capability module by eliminating rounds with an rₛ value of less than 0.60 among the m rounds of ranking by each sensory assessor. Then, the rank data rₛ value for each remaining round of ranking is converted into a corresponding equidistant data Zᵣ value using Zᵣ Fisher conversion, and arithmetic mean value Z̅ᵣ from the Zᵣ values of the remaining rounds is obtained after eliminating the abnormal experiments for each sensory assessor, where a higher Z̅ᵣ value signifies a more correct ranking capability. The Zᵣ Fisher conversion is used to convert the rₛ value for each ranking experiment by each sensory assessor into a Zᵣ value according to the following calculation formula:

$${Z}_{r} = {tanh}^{- 1} ( {r}_{s} ) = {\sum }_{N = 0}^{∞} \frac{{r}_{s}^{2 N + 1}}{2 N + 1} \qquad ( 2 )$$

where rₛ is the rank correlation coefficient, and N is the number of inverse hyperbolic tangent expansion terms.

The value of Z̅ᵣ is calculated according to the following formula:

$$\overline{{Z}_{r}} = \frac{{\sum }_{j = 1}^{m} ( {n}_{j} - 3 ) {Z}_{rj}}{{\sum }_{j = 1}^{m} ( {n}_{j} - 3 )} \qquad ( 3 )$$

where m is the number of evaluation repeats after eliminating any abnormal experiments; nⱼ is the number of samples in the j^{th} repeated evaluation, and nⱼ=6; Zrⱼ value is the Fisher conversion Zr value of the correlation coefficient rₛ in the j^{th} repeated evaluation.

Then, the repeated ranking capability of a sensory assessor is assessed using the repeated ranking capability module by calculating an S_{Zr} of the Zᵣ values for the remaining rounds obtained after eliminating abnormal experiments for each sensory assessor. The repeated ranking capability of each sensory assessor is reflected according to the S_{Zr}. Therefore, the smaller the S_{Zr}, the higher the repeated ranking capability.

S_{Zr} is calculated according to the following formula:

$${S}_{{Z}_{r}} = \sqrt{\frac{{\sum }_{j = 1}^{m} {( {Z}_{{r}_{j}} - \overline{{Z}_{r}} )}^{2}}{m}} \qquad ( 4 )$$

where m is the number of evaluation repeats after eliminating abnormal experiments; Zrⱼ is the Fisher conversion Zᵣ of the correlation coefficient rₛ in the j^{th} repeated evaluation; Z̅ᵣ is a mean value of the Zᵣ values obtained by applying the Fisher conversion to the rₛ values of the remaining rounds after eliminating the abnormal experiments for a particular assessor.

Then, a ratio (CV value) of the S_{Zr} of the Zᵣ values is calculated after multiple rounds of ranking to obtain Z̅ᵣ for each sensory assessor using the ranking focusing capability module. The CV value is calculated according to the following formula:

$$CV = \frac{{S}_{{Z}_{r}}}{\overline{{Z}_{r}}} . \qquad ( 5 )$$

The second kind of data refers to the median and mode of the rₛ value; the third kind of data. is the Z̅ᵣ value: the fourth kind of data is the S_{Zr} value, and the fifth kind of data is the CV value.

Finally, the data analysis unit is configured to analyze the CV value of each sensory assessor group; a CV value of >20% denotes the first sensory assessor group (with excellent ranking capability) possessing high sensibility and poor attention; a CV value of ≤17% denotes the second sensory assessor group (with good ranking capability) possessing moderate sensibility and high attention, and a CV value of ≤21% signifies the third sensory assessor group (with poor ranking capability) possessing fair sensibility and high attention.

The serial numbers for a sensory assessor turn red in the result display module when they display high sensibility and poor attention, yellow in the case of moderate sensibility and high attention, green in the case of fair sensibility and high attention.

### Embodiment 2

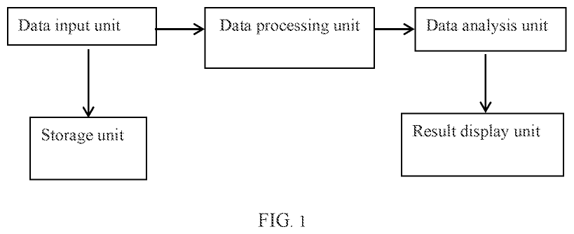

FIG. 1 shows the system for assessing the concentration ability of sensory assessors and comprises of the following steps: a data input unit for entering the first kind of data; a storage unit for storing the first kind of data; a data processing unit for processing the first kind of data to obtain the second kind of data, the third kind of data, the fourth kind of data, and the fifth kind of data; a data analysis unit for analyzing the second kind of data, the third kind of data, the fourth kind of data and the fifth kind of data, thereby providing the concentration ability of a sensory assessor; and a result display unit for displaying the serial numbers denoting the concentration ability of the sensory assessors.

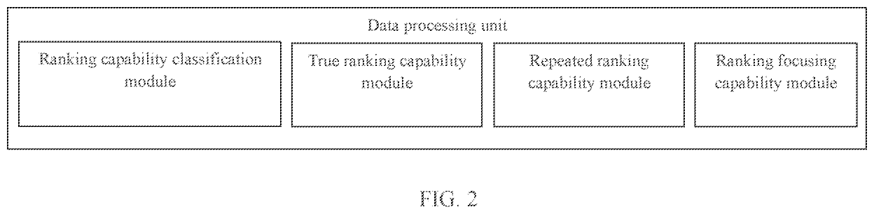

The data processing unit includes a ranking capability classification module, a true ranking capability module, a repeated ranking capability module, and a ranking focusing capability module (as shown in FIG. 2).

The ranking capability classification module is configured to calculate a Spearman rank correlation coefficient rₛ of each ranking result for each sensory assessor according to the ranking information. Then, statistical analysis is performed to calculate a median and mode of the Spearman rank correlation coefficient rₛ obtained after m rounds of ranking experiments for each sensory assessor are calculated.

The correct ranking capability module is configured to evaluate the correct ranking capability of a sensory assessor by eliminating a result for a round with an r value of less than 0.60 among the in rounds of ranking by each sensory assessor. The rank data rₛ value for each remaining round of ranking is converted into a corresponding equidistant data Zᵣ value via Zᵣ Fisher conversion, and then an arithmetic mean value Z̅ᵣ of the Zᵣ values for the remaining rounds are obtained after eliminating the abnormal experiments for each sensory assessor. Therefore, the greater the Z̅ᵣ, the higher the correct ranking capability.

The repeated ranking capability module is configured to evaluate the repeated ranking capability of a sensory assessor by calculating an S_{Zr} of the Zᵣ values for the remaining rounds, obtained after eliminating the abnormal experiments for each sensory assessor. Then, the repeated ranking capability of each sensory assessor is reflected according to the S_{Zr}, showing that a smaller the S_{Zr} induces a higher repeated ranking capability.

The ranking focusing capability module is configured to calculate the ratio (CV value) of the S_{Zr} of the Zᵣ values to the Z̅ᵣ value for each sensory assessor after multiple rounds of ranking.

The data analysis unit is configured to analyze the CV value of each sensory assessor group. A CV value of >20% denotes the first kind of sensory assessor group (with excellent ranking capability) possessing high sensibility and poor attention; a CV value of ≤17% denotes the second kind of sensory assessor group (with good ranking capability) possessing moderate sensibility and high attention, and a CV value of ≤21% signified the third kind of sensory assessor group (with poor ranking capability) possessing fair sensibility and high attention.

The serial numbers for sensory assessors turn red in the result display module when they display high sensibility and poor attention, yellow in the case of moderate sensibility and high attention, green in the case of fair sensibility and high attention.

Although the present invention has been presented explicitly via the general description and detailed embodiments mentioned above, it will be apparent to those skilled in the art that some modifications or improvements can he made based on the present invention. However, making these modifications or improvements should not depart from the spirit of the present invention and must remain within its protection scope.

What is claimed is:

1. A method for evaluating a concentration ability of sensory assessors, comprising:
S1, entering a first kind of data to a data input unit and saving the first kind of data. to a storage unit;
S2, processing the first kind of data with a data processing unit to obtain a second kind of data, a third kind of data, a fourth kind of data, and fifth kind of data;
S3, analyzing the second kind of data, the third kind of data, the fourth kind of data and the fifth kind of data with a data analysis unit, to determine the concentration ability of each of the the sensory assessors; and
S4, displaying a serial number relating to the concentration ability of each of the sensory assessors in a result display unit;
wherein the first kind of data are obtained by repeatedly ranking n samples at different concentrations on sensory quality for m rounds of ranking by each of the sensory assessors, wherein n=6 and m=12:
wherein the data processing unit comprises a ranking capability classification module, a true ranking capability module, a repeated ranking capability module, and a ranking focusing capability module:
wherein the value of a Spearman rank correlation coefficient rₛ for each of the m rounds of ranking by each of the sensory assessors is calculated using the ranking capability classification module according to the ranking information: then, a median and a mode of the values of the Spearman rank correlation coefficients rₛ are obtained after the m rounds of ranking by each of the sensory assessors are calculated:
the true ranking capability module evaluates the correct ranking capability of each of the sensory assessors after eliminating abnormal results for a round with an rₛ value of less than 060 among the in rounds of ranking by each of the sensory assessors: the rₛ value for each remaining round of ranking is converted into a corresponding equidistant data Zᵣ value via a Zᵣ Fisher conversion, and an arithmetic mean value Z̅ᵣ of the Zᵣ values is calculated for the remaining rounds obtained after eliminating the round with the abnormal result for each of the sensory assessors, indicating that a higher Z̅ᵣ value induces a more true ranking capability;
the repeated ranking capability module evaluates a repeated ranking capability of each of the sensory assessors: calculating a S_{Zr} of the Zᵣ values for the remaining rounds obtained after kicking out the round with the abnormal result for each of the sensory assessors; and the repeated ranking capability of each of the sensory assessors is reflected according to the S_{Zr}, wherein the smaller the S_{Zr} is, the higher the repeated ranking capability is:
a ratio of the S_{Zr} of the Zᵣ values to the Z̅ᵣ values for each of the sensory assessors after the m rounds of ranking is calculated using the ranking focusing capability module and the ratio calculated is a CV (Coefficient of Variation) value, wherein the CV value is calculated according to the following formula:
$$CV = \frac{{S}_{{Z}_{r}}}{\overline{{Z}_{r}}} ; \qquad ( 1 )$$
wherein the second kind of data is represented by the median and the mode of the rₛ values; the third kind of data is the Z̅ᵣ value: the fourth kind of data is the S_{Zr} value, and the fifth kind of data is the CV value:
wherein when n=6, the data analysis unit is configured to analyze the second kind of data, wherein a sensory assessor with a mode=1.00 belongs to the first kind of sensory assessor group exhibiting an excellent ranking capability, while a sensory assessor with a median=0.943 belongs to the second kind of sensory assessor group displaying a good ranking capability, and the remaining sensory assessors belong to the third kind of sensory assessor group displaying a poor ranking capability;
the data analysis unit is configured to analyze the CV value of each sensory assessor group, wherein, a CV value of >20% denotes the first kind of sensory assessor group possessing a high sensibility and a poor attention, a CV value of ≤17% denotes the second kind of sensory assessor group possessing a moderate sensibility and a high attention, and a CV value of ≤21% signifies the third kind of sensory assessor group possessing a fair sensibility and the high attention.

2. (canceled)

3. (canceled)

4. (canceled)

5. The method for evaluating the concentration ability of the sensory assessors according to claim 1, wherein the Spearman rank correlation coefficient rₛ is calculated according to the following formula:
$${r}_{s} = 1 - \frac{6 {\sum }_{i = 1}^{n} {d}_{i}^{2}}{n ( {n}^{2} - 1 )} [ [ ( 1 ) ] ] , \qquad ( 2 )$$
wherein the rₛ is Spearman the rank correlation coefficient; n is a number of ranking experiment samples; dᵢ is a difference between a real rank and a rank of a sensory assessor of an i^{th} sample in a round of ranking.

6. The method for evaluating the concentration ability of the sensory assessors according to claim 1, wherein the Zᵣ Fisher conversion is used to convert the rₛ value for each ranking round by each of the sensory assessors into the Zᵣ value according to the following calculation formula:
$${Z}_{r} = {tanh}^{- 1} ( {r}_{s} ) = {\sum }_{N = 0}^{∞} \frac{{r}_{s}^{2 N + 1}}{2 N + 1} [ [ ( 2 ) ] ] , \qquad ( 3 )$$
wherein rₛ is the Spearman rank correlation coefficient; and N is a number of inverse hyperbolic tangent expansion terms;
the value of Z̅ᵣ is calculated according to the following formula:
$$\overline{{Z}_{r}} = \frac{{\sum }_{j = 1}^{m} ( {n}_{j} - 3 ) {Z}_{rj}}{{\sum }_{j = 1}^{m} ( {n}_{j} - 3 )} [ [ ( 3 ) ] ] . \qquad ( 4 )$$
wherein m is a number of evaluation repeats after eliminating the abnormal result ; nⱼ is a number of samples in a j^{th} repeated evaluation, and nⱼ:=6; the Zᵣⱼ value is the Fisher conversion Zᵣ value of the Spearman correlation coefficient rₛ value for the j^{th} repeated evaluation where the S_{Zr} is calculated according to the following formula:
$${S}_{{Z}_{r}} = \sqrt{\frac{{\sum }_{j = 1}^{m} {( {Z}_{{r}_{j}} - \overline{{Z}_{r}} )}^{2}}{m}} [ [ ( 4 ) ] ] , \qquad ( 5 )$$
wherein m is the number of evaluation repeats after eliminating the round with the abnormal result; Zᵣⱼ is the Fisher conversion Zᵣ value of the Spearman correlation coefficient rₛ value in the j^{th} repeated evaluation; Z̅ᵣ is a mean value of the Zᵣ values obtained by applying the Fisher conversion to the rₛ value of the remaining rounds after eliminating the round with the abnormal result for a sensory assessor.

7. (canceled)

8. The method for evaluating the concentration ability of the sensory assessors according to claim 1, wherein the serial number for each of the sensory assessors turn red in the result display unit when the sensory assessors display the high sensibility and the poor attention, yellow for the moderate sensibility and the high attention, and green in the case for the fair sensibility and the high attention.

9. A system for evaluating the concentration ability of the sensory assessors while using the method according to claim 1, comprising:
the data input unit for entering the first kind of data;
the storage unit for storing the first kind of data;
the data processing unit for processing the first kind of data to obtain the second kind of data, the third kind of data, the fourth kind of data, and the fifth kind of data;
the data analysis unit for assessing the second kind of data, the third kind of data, the fourth kind of data, and the fifth kind of data to determine the concentration ability of each of the sensory assessors; and
the result display unit for displaying the serial number representative of the concentration ability of each of the sensory assessors.

10. A device comprising the system according to claim 9.

11. The system according to claim 9, wherein the Spearman rank correlation coefficient rₛ is calculated according to the following formula:
$${r}_{s} = 1 - \frac{6 {\sum }_{i = 1}^{n} {d}_{i}^{2}}{n ( {n}^{2} - 1 )} \qquad ( 2 )$$
wherein the rₛ is Spearman the rank correlation coefficient; n is a number of ranking experiment samples; dᵢ is a difference between a real rank and a rank of a sensory assessor of an i^{th} sample in a round of ranking.

12. The system according to claim 9, wherein the Zr Fisher conversion is used to convert the rₛ value for each ranking round by each of the sensory assessors into the Zᵣ value according to the following calculation formula:
$${Z}_{r} = {tanh}^{- 1} ( {r}_{s} ) = {\sum }_{N = 0}^{∞} \frac{{r}_{s}^{2 N + 1}}{2 N + 1} , \qquad ( 3 )$$
wherein rₛ is the Spearman rank correlation coefficient; and N is a number of inverse hyperbolic tangent expansion terms;
the value of Zᵣ is calculated according to the following formula:
$$\overline{{Z}_{r}} = \frac{{\sum }_{j = 1}^{m} ( {n}_{j} - 3 ) {Z}_{rj}}{{\sum }_{j = 1}^{m} ( {n}_{j} - 3 )} , \qquad ( 4 )$$
wherein m is a number of evaluation repeats after eliminating the abnormal result; nⱼ is a number of samples in a j^{th} repeated evaluation, and nⱼ=6; the Zᵣⱼ value is the Fisher conversion Zᵣ value of the Spearman correlation coefficient rₛ value for the j^{th} repeated evaluation where the S_{Zr} is calculated according to the following formula:
$${S}_{{Z}_{r}} = \sqrt{\frac{{\sum }_{j = 1}^{m} {( {Z}_{{r}_{j}} - \overline{{Z}_{r}} )}^{2}}{m}} , \qquad ( 5 )$$
wherein m is the number of evaluation repeats after eliminating the round with the abnormal result; Zᵣⱼ is the Fisher conversion Zᵣ value of the Spearman correlation coefficient rₛ value in the j^{th} repeated evaluation; Z̅ᵣ, is a mean value of the Zᵣ values obtained by applying the Fisher conversion to the rₛ value of the remaining rounds after eliminating the round with the abnormal result for a sensory assessor.

13. The system according to claim 9, wherein the serial number for each of the sensory assessors turn red in the result display unit when the sensory assessors display the high sensibility and the poor attention, yellow for the moderate sensibility and the high attention, and green for the fair sensibility and the high attention.
